(19) United States
(12) Patent Application Publication
(10) Pub. No.: US 2015/0328266 A1
(43) Pub. Date: Nov. 19, 2015

(54) Use of Bifidobacterium Animalis for Treating or Preventing Body Weight Gain and Insulin Resistance

(71) Applicants: COMPAGNIE GERVAIS DANONE, Paris (FR); TUFTS UNIVERSITY, Boston, MA (US)

(72) Inventors: Jian Shen, Shanghai (CN); JingJing Wang, Shanghai (CN); Liping Zhao, Shanghai (CN); Martin Saul Obin, West Newton, MA (US); Muriel Derrien, Bures Sur Yvette (FR); Emilie Rocher, Massy (FR); Johan Van Hylckama Vlieg, Marly le Roi (FR)

(73) Assignees: TUFTS UNIVERSITY, Boston, MA (US); COMPAGNIE GERVAIS DANONE, Paris (FR)

(21) Appl. No.: 14/653,546

(22) PCT Filed: Dec. 20, 2012
(86) PCT No.: PCT/CN2012/087043

§ 371 Date: Jun. 18, 2015

(51) Int. Cl. A61K 35/745; A23L 1/30; A23C 9/12; A23C 9/123

(52) U.S. Cl. CPC A61K 35/745; A23C 9/123; A23L 1/3014; A23C 9/1203; A61K 2035/115

(57) ABSTRACT

Provided is the use of Bifidobacterium animalis subsp. lactis strain CNCM I-2494 for decreasing diet-induced body weight gain and improving diet-induced insulin resistance in a subject.

The present invention relates to the use of probiotic bacteria for preventing or treating high diet-induced obesity and insulin resistance. Particularly, the present invention relates to a composition comprising a bacterial strain of the Bifidobacterium animalis subsp. lactis species intended for decreasing the body weight gain and improving the insulin resistance in a subject.

The worldwide prevalence of overweight, obesity and insulin resistance, which are crucial risk factors for diabetes and cardiovascular disease (Alberti et al., 2005), are thought to be resulted from the excess intake of high fat/calorie diet and or reduced physical exercise (Vijay-Kumar et al., 2010).

A body mass index (BMI; kg/m²) greater than or equal to 25 is considered overweight and a BMI greater or equal to 30 is defined as obesity.

Obesity is often associated with insulin resistance (i.e. a condition where cells are no longer able to respond adequately to insulin) leading to major diseases that encompass metabolic syndrome such as hypertension, type II diabetes, cardiovascular diseases, as well as liver diseases.

Overweight, obesity, diabetes and related metabolic diseases are characterized by low-grade and chronic inflammation in circulating system and tissues.

The insulin signaling is a complex system, and a common mechanism to explain the occurrence of acute (mediated, at least in part, by the action of pro-inflammatory cytokines) and chronic (mediated by genetic variation due to aging and obesity) insulin resistance is difficult to identify (Aguirre et al., 2002).

Recent research have shown that gut microbiota plays a trigger role in the high fat diet (HFD)-induced obesity (Ley et al., 2006; Turnbaugh et al., 2006) and insulin resistance (Cani et al., 2008; Larsen et al., 2010). The gut microbiota plays a role in the digestion of indigestible food components, regulates host fat storage genes, and then modulates host energy homeostasis (Bäckhed et al. 2004 and 2007). The disrupted gut microbiota by HFD increases intestinal permeability. Consequently, increased levels of endotoxin from the gut bacteria enter the circulating system, and provoke inflammation, which may induce obesity and insulin resistance (Cani et al., 2008). Therefore, gut microbiota could be a potential target of prevention and treatment of obesity and insulin resistance (Jia et al., 2008; Zhao et al., 2010).

According to the currently adopted definition by FAO/WHO, probiotics are live microorganisms which when administered in adequate amounts confer a health benefit to the host. Particularly, according to a definition approved by the National Yogurt Association (NYA) or the International Life Science Institute (ILSI) in the USA, probiotics are living micro-organisms which upon ingestion in a sufficient amount exert health benefits beyond basic nutrition. Probiotic bacteria have been described among species belonging to the genera Lactobacillus, Bifidobacterium, Streptococcus and Lactococcus, commonly used in the dairy industry. Oral consumption of probiotics can change the structure of gut microbiota. By way of example, the amount of Lactobacillus and Bifidobacterium in the gut of a subject is higher after intake of some probiotics by said subject (Xu et al., 2012). Consumption of fermented milk product comprising probiotics probably does not induce a major change in the bacterial species composition in the gut, but significant changes expression of microbiome-encoded enzymes involved in carbohydrate metabolism (McNulty et al., 2011). Some probiotics decrease HFD-induced obesity (Lee et al., 2006; Yin et al., 2010), improve insulin resistance (Andreasen et al. 2010) or show anti-inflammatory properties (Menard et al., 2004; Andreasen et al., 2010; Veiga et al., 2010; Fernandez et al., 2011).

However, the probiotic-induced bacterial changes that are closely associated with metabolic disease remain unclear. Further, different probiotic strains show different functions and mechanisms.

Bifidobacterium animalis (B. animalis) is a Gram-positive anaerobic rod-shaped bacterium, which can be found in the large intestines of most mammals, including humans. Bifidobacterium animalis and Bifidobacterium lactis were previously described as two distinct species. Presently, both are considered Bifidobacterium animalis with the subspecies animalis and lactis, respectively. Both old names Bifidobacterium animalis and Bifidobacterium lactis are still used on product labels, as this species is frequently used as a probiotic. The names Bifidobacterium lactis and Bifidobacterium animalis subsp. lactis can be used interchangeably.

It has previously been shown that some strains of Bifidobacterium animalis subsp. lactis have a glycosylation modulating effect of intestinal cell surface (International Application WO 02/02800), decrease boborygmi (International Application WO 2009/150036), decrease abdominal girth (International Application WO 2009/080800), lower cecal pH and alter short chain fatty acid profiles, then inhibiting the growth of pathogenic bacteria in the mice with colitis (Veiga et al., 2010), reduce gastro-intestinal inflammation (International Application WO 2011/051760), and suppress intestinal mucosal adherence and translocation of commensal bacteria to treat type 2 diabetes (Amar et al., 2011). International Application WO 2010/146568 discloses the use of the Bifidobacterium animalis subsp. lactis strain 420 (B420) for treating obesity, controlling weight gain, inducing weight loss, treating diabetes, normalising insulin sensitivity and treating metabolic syndrome.

The effects of these different probiotics are strain-specific, and appear to be mediated by different mechanisms. Thus, a need remains for other probiotic strains that can be used for controlling the development of overweight and obesity and metabolic diseases associated therewith.

The inventors have undertaken to study the preventive effects of probiotics on HFD-induced obesity and insulin resistance in mice. It is well known that high fat diet induces in mice or human body weight gain and insulin resistance. The inventors have shown that Bifidobacterium animalis subsp. lactis strain CNCM I-2494 orally administrated to high fat diet (HFD)-fed mice at 10⁸ cells/day for 12 weeks, significantly reduced body weight gain and improved insulin resistance. Compared with the B. animalis subsp. lactis strain 420 (B420) that also showed anti-inflammation tendency, B. animalis subsp. lactis strain CNCM I-2494 most effectively reduced systemic antigen load, and local inflammation in liver, epididymal adipose tissue and jejunum in high fat diet-fed mice. Principal component analysis (PCA) analysis on 454 pyrosequencing data of fecal bacterial 16S rRNA genes showed that B. animalis subsp. lactis strain CNCM I-2494 changes the structure of gut microbiota. Partial least square discriminate analysis (PLS-DA) revealed that B. animalis subsp. lactis strain CNCM I-2494 also changes the relative abundance of different operational taxonomic units (OTUs), but most elevated OTUs were from lactate and acetate-producing bacteria. One OTU from Porphyromonadaceae, which is significantly associated with inflammatory parameters, was specifically changed by B. animalis subsp. lactis strain CNCM I-2494, while it is not changed by B. animalis subsp. lactis strain 420. These results suggest that prevention of obesity and insulin resistance by B. animalis subsp. lactis strain CNCM I-2494 is associated with changes in lactate and acetate-producing bacteria, and alleviation of inflammation is associated with Porphyromonadaceae.

Bifidobacterium animalis subsp. lactis CNCM I-2494 was deposited according to the Budapest Treaty with the CNCM on Jun. 20, 2000. This strain is known under the code DN 173 010 and was first disclosed in International Application WO 02/02800 for use as glycosylation modulator of gastro-intestinal cell surface.

Accordingly, an object of the present invention is the Bifidobacterium animalis subsp. lactis strain CNCM I-2494 or a composition comprising said strain CNCM I-2494 for use for decreasing diet-induced body weight gain and improving diet-induced insulin resistance in a subject.

Said Bifidobacterium animalis subsp. lactis strain CNCM I-2494 or said composition comprising said strain CNCM I-2494 is further for use for alleviating inflammation.

This alleviation of inflammation is associated with an enhancement of Porphyromonadaceae in the gut of said subject.

The inflammation is preferably localized in liver, epididymal adipose tissue and/or jejunum of said subject.

“Diet-induced body weight gain” and “diet-induced insulin resistance” are defined herein as body weight gain and insulin resistance resulting from an excessive dietary intake, including an excessive dietary intake of fat, in particular unsaturated fat, and optionally an excessive dietary intake of simple sugars, including sucrose and fructose. For a given subject, an excessive dietary intake, in particular of fat and optionally of simple sugars, refers to the consumption of an amount of diet, in particular of fat and optionally of simple sugars, higher than the amount necessary to meet the physiological needs and maintain the energy balance of said subject. The effect of a treatment on reduction of—or prevention—of diet-induced body weight gain and insulin resistance in a subject can be assessed by comparing body weight gain and insulin resistance observed in a subject receiving the treatment with those observed in the same subject without treatment receiving the same diet and having the same level of physical activity.

As used herein, “decreasing the body weight gain” means limiting, lowering or reducing the enhancement of body weight induced by a given diet as defined above in a subject by comparison to the enhancement of body weight induced by said given diet in said subject but who would not consume the B. animalis subsp. lactis strain CNCM I-2494.

As used herein, “improving the insulin resistance” means ameliorating or decreasing the level of insulin resistance induced by a given diet as defined above in a subject by comparison to the level of insulin resistance induced by said given diet in said subject but who would not consume the B. animalis subsp. lactis strain CNCM I-2494.

Tests for evaluating insulin resistance in a subject are known in the art (see for review Ferrannini et al., 1998). The level of insulin resistance in a subject can be measured with any insulin resistance test known in the art, such as the homeostatic model assessment of insulin resistance (HOM-IR).

In a preferred embodiment of the present invention, the body weight gain and insulin resistance are induced by (i.e., associated to) a high fat diet (HFD) in said subject.

Determining the alleviation of inflammation, in particular in liver, epididymal adipose tissue and/or jejunum from a subject, can be carried out by measuring TNF-α, CD11c, MCP-1, adiponectin and leptin mRNA expression. A method is described in the Example below.

The present invention also encompasses the Bifidobacterium animalis subsp. lactis strain CNCM I-2494 or a composition containing said strain, for use in the treatment, prevention, or alleviation of a condition resulting from diet-induced body weight gain and diet-induced insulin resistance, as defined above, in a subject.

Examples of conditions resulting from diet-induced weight gain and diet-induced insulin resistance are overweight, obesity, and related disorders, such as type 2 diabete, non-alcoholic fatty liver disease (NAFLD), hypertension.

A subject of the present invention in also the use of the Bifidobacterium animalis subsp. lactis strain CNCM I-2494 as a compound for decreasing diet-induced body weight gain and improving diet-induced insulin resistance, and optionally for alleviating inflammation, in a subject as defined above, in a nutritional composition.

The composition of the present invention can be in any form suitable for administration, in particular oral administration. This includes for instance solids, semi-solids, liquids, and powders. Liquid composition are generally preferred for easier administration, for instance as drinks.

In the composition of the invention, said bacterial strain can be used in the form of whole bacteria which may be living or dead. Alternatively, said strain can be used in the form of a bacterial lysate. Preferably, the bacterial strain is present as living, viable cell.

When said strain CNCM I-2494 is in the form of living bacterium, the composition may typically comprise 10⁵ to 10¹³ colony forming units (cfu), preferably at least 10⁶ cfu, more preferably at least 10⁷ cfu, still more preferably at least 10⁸ cfu, and most preferably at least 10⁹ cfu per g dry weight of the composition. In the case of a liquid composition, this corresponds generally to 10⁴ to 10¹² colony forming units (cfu), preferably at least 10⁵ cfu, more preferably at least 10⁶ cfu, still more preferably at least 10⁷ cfu, and most preferably at least 10⁹ cfu/ml.

Said CNCM I-2494 may be used alone, or in combination with other lactic acid bacteria of the Bifidobacterium animalis subsp. lactis species or of other species. Advantageously, it may be used in combination with yogurt ferments, namely Lactobacillus bulgaricus and Streptococcus thermophilus.

When said strain CNCM I-2494 is used in combination with yogurt ferments, said composition also advantageously comprises at least 10⁷, preferably between 2×10⁸ and 1×10⁹ S. thermophilus cells per ml, and at least 5×10⁵ and preferably between 4×10⁶ and 2×10⁷ L. bulgaricus cells per ml.

The composition according to the present invention includes food products, food supplements and functional food.

A “food supplement” designates a product made from compounds usually used in foodstuffs, but which is in the form of tablets, powder, capsules, potion or any other form usually not associated with aliments, and which has beneficial effects for one's health.

A “functional food” is an aliment which also has beneficial effects for one's health. In particular, food supplements and functional food can have a physiological effect —protective or curative—against a disease, for example against a chronic disease.

The composition of the invention also includes a baby food, an infant milk formula or an infant follow-on formula. The present composition can also be a nutraceutical, a nutritional supplement or medical food.

The composition of the invention can be a dairy product, preferably a fermented dairy product. The fermented product can be present in the form of a liquid or present in the form of a dry powder obtained by drying the fermented liquid. Examples of dairy products include fermented milk and/or fermented whey in set, stirred or drinkable form, cheese and yoghurt.

The fermented product can also be a fermented vegetable, such as fermented soy, cereals and/or fruits in set, stirred or drinkable forms.

In a preferred embodiment, the fermented product is a fresh product. A fresh product, which has not undergone severe heat treatment steps, has the advantage that the bacterial strains present are in the living form.

The composition may, for example, be a milk product, and in particular a fermented milk product comprising at least said strain CNCM I-2494, optionally combined, as indicated above, with other lactic acid bacteria, for example with yogurt ferments.

The amount of said strain CNCM I-2494 administered daily will preferably be at least 2×10³, advantageously at least 2×10⁸ and more advantageously at least 2×10¹⁰ CFU. This amount can be administered in one or more daily intakes during the high fat diet. In order to obtain an optimal effect, said strain CNCM I-2494 will preferably be administered twice a day during the high fat diet.

A subject of the present invention is also the Bifidobacterium animalis subsp. lactis strain CNCM I-2494, for use as a pharmaceutical composition, preferably a pharmaceutical nutritional composition as defined above, for decreasing diet-induced body weight gain and improving diet-induced insulin resistance, and optionally for alleviating inflammation, in a subject as defined above.

A subject of the present invention is also a method for decreasing diet-induced body weight gain and improving diet-induced insulin resistance, and optionally for alleviating inflammation, as defined above in a subject in need thereof, wherein said method comprises administrating to said subject a therapeutically effective amount of the Bifidobacterium animalis subsp. lactis strain CNCM I-2494 or a composition containing said strain.

Determination of a therapeutically effective amount is well known from the person skilled in the art, especially in view of the detailed disclosure provided herein.

The term “administering” is intended to mean “administering orally” i.e. that the subject will orally ingesting the bacterial strain according to the present invention or a composition comprising the bacterial strain according to the present invention, or is intended to mean “administering directly” i.e. that a bacterial strain according to the present invention or a composition comprising the bacterial strain according to the present invention will be directly administered in situ, in particular by coloscopy, or rectally via suppositories.

Oral administration of the composition comprising the bacterial strain according to the present invention is preferred. It may be in the form of gelatin capsules, capsules, tablets, powders, granules or oral solutions or suspensions.

The present invention will be understood more clearly from the further description which follows, which refers to examples illustrating the effect of the Bifidobacterium animalis subsp. lactis strain CNCM I-2494 on the decrease of body weight gain and the improvement of insulin resistance induced by a high fat diet in mice as well as to the appended figures.

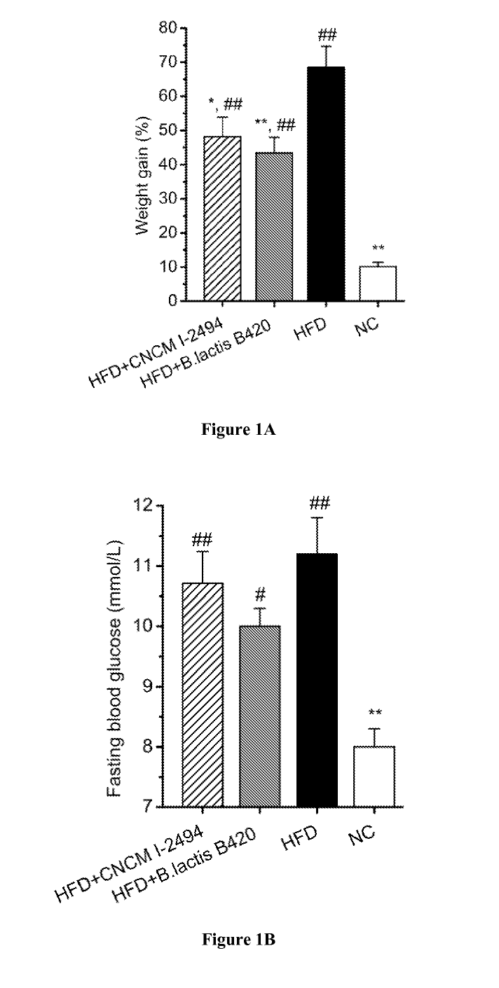

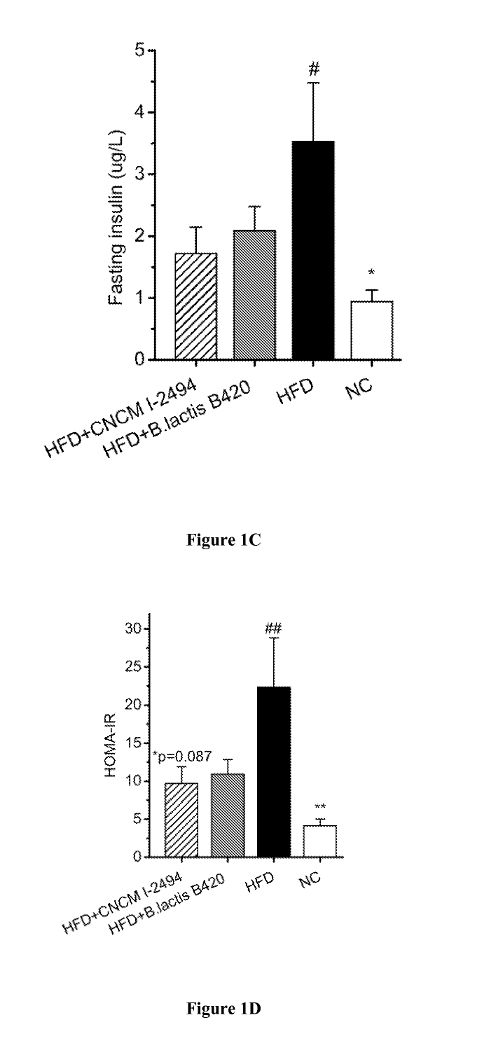

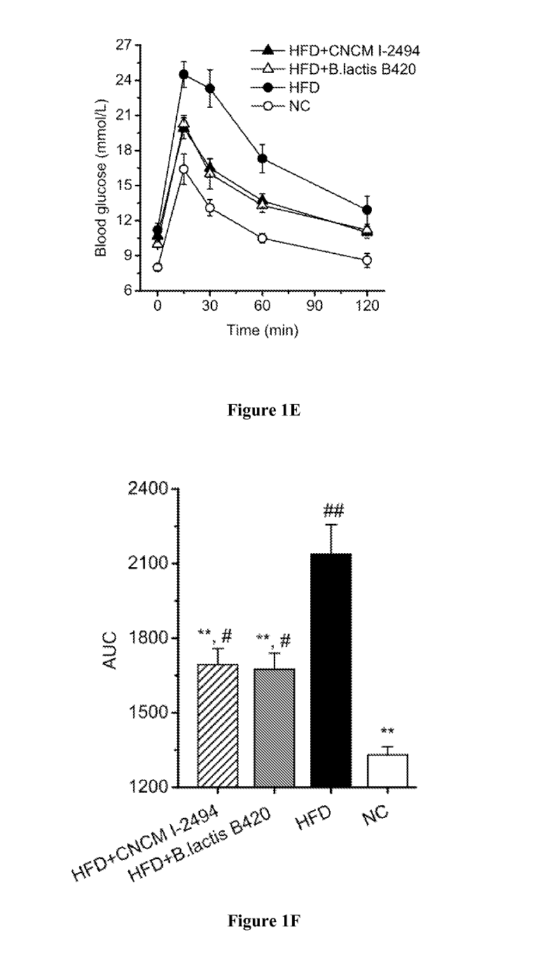

FIG. 1: Weight gain (A), fasting blood glucose (B), fasting insulin (C), HOMA-IR (D), OGTT (E) and areas under the curve (AUC) of OGTT (F) for four groups: NC (normal chow), HFD (high fat diet), HFD+CNCM I-2494, HFD+B. lactis B420. Data are shown as means±S.E.M. **p<0.01, *p<0.05 when compared to HFD group, and ##p<0.01, #p<0.05 when compared to NC group by One Way-ANOVA followed by Tukey post hoc test in SPSS. HOMA-IR is calculated according to the following formula: fasting blood glucose (mmol/L)×fasting insulin (mU/L)/22.5.

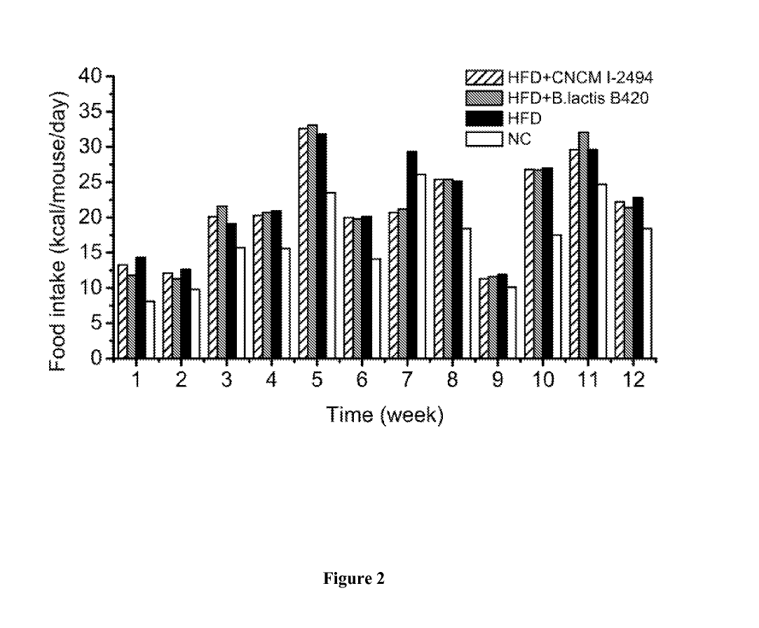

FIG. 2: Food intake of the NC, HFD, HFD+CNCM I-2494 and HFD+B. lactis B420 groups each week. Data are shown as means of two cages of mice. The statistical analysis was not performed.

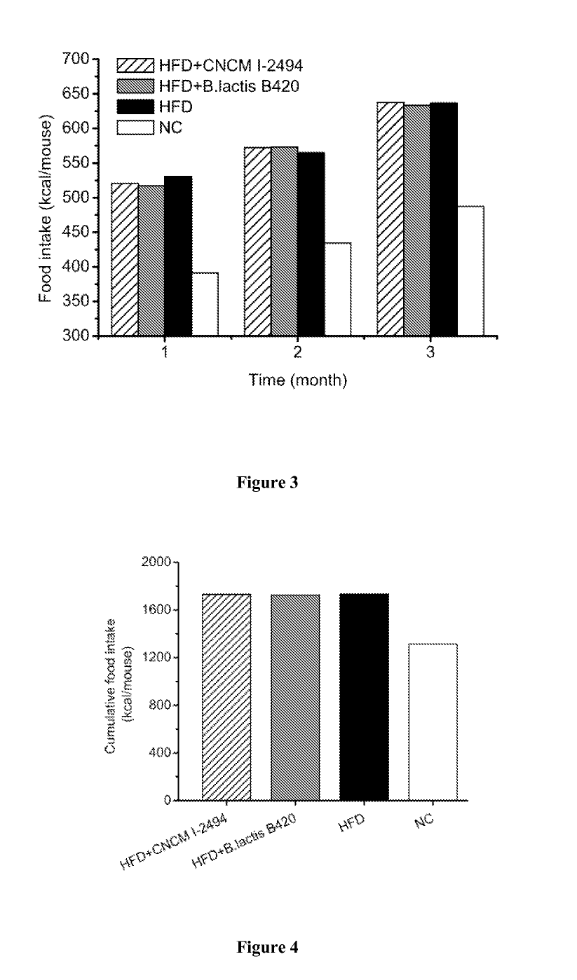

FIG. 3: Cumulative food intake of the NC, HFD, HFD+CNCM I-2494 and HFD+B. lactis B420 groups each month of the animal trial. Data are shown as means of two cages of mice. The statistical analysis was not performed.

FIG. 4: Cumulative food intake of the NC, HFD, HFD+CNCM I-2494 and HFD+B. lactis B420 groups during 12 weeks. Data are shown as means of two cages of mice. The statistical analysis was not performed.

### EXAMPLE

### Decrease of High Fat Diet-Induced Body Weight Gain and Improvement of High Fat Diet-Induced Insulin Resistance by Bifidobacterium animalis Subsp. Lactis Strain CNCM I-2494 in Mice

### Materials and Methods

Animal Treatment

C57BL/6J mice (male, at age 12 weeks) were divided into 3 groups (8 mice per group) under different treatments as follows:

Group A: high fat diet, containing 34.9% fat, 5.24 kcal/g, from Research Diets, Inc., New Brunswick, N.J. (HFD);

Group B: high fat diet, plus probiotic strain Bifidobacterium animalis subsp. lactis strain CNCM I-2494, at 10⁸ CFU/mouse/day (HFD+CNCM I-2494);

Group C: high fat diet, plus probiotic strain Bifidobacterium animalis subsp. lactis B420 (Danisco), at 10⁸ CFU/mouse/day (HFD+B. lactis B420), previously reported to reduce adverse effects on metabolism associated with high-fat diet (Amar et al., 2011, cited above), as a comparison strain;

Group D: normal chow, containing 4.3% fat, 3.85 kcal/g, from Research Diets, Inc., New Brunswick, N.J. (NC).

B. lactis CNCM I-2494 or B. lactis B420 suspension were prepared before the animal trial, stored at −80° C. and thawed 1 hour before they were administered to each mouse by oral feeding.

Animal treatments lasted for 12 weeks, during which the body weight of each mouse and food intake of every cage of mice were measured twice a week. Fresh stool and urine samples were collected once a month by using a metabolic cage and immediately stored at −80° C. for subsequent analysis.

The amount of the probiotic strains in the feces of mice at 2^{nd}, 6^{th} and 11^{th} weeks during the probiotic administration was quantified by reverse transcription (RT)-qPCR, and the results confirmed that they could survive in the intestine.

At the end of the trial, after 5 h of food deprivation, blood was collected from the orbital plexus, and serum was isolated by centrifugation at 3000 rpm at 4° C. for 15 min. All animals were sacrificed by cervical dislocation. Epididymal fat pads, liver and jejunum were excised, weighed, and immediately kept in RNALater (Ambion) after sacrifice.

Oral Glucose Tolerance Test (OGTT)

Oral glucose tolerance tests (OGTT) were performed before the sacrifice of animals. After 5 h of food deprivation, 2.0 g/kg body weight glucose was administered orally to the mice. Blood samples were taken from the tail to measure blood glucose levels before and 15, 30, 60, and 120 min after glucose administration by using an ACCU-Check glucose meter (Roche Diagnostics, Canada).

The blood glucose level before glucose administration is regarded as fasting blood glucose (FBG) level.

Fasting Insulin, LBP and Adiponectin Levels

Fasting insulin (FINS), lipopolysaccharide-binding protein (LBP) and adiponectin levels were determined by ELISA assays (respectively Mercodia, Sweden; Cell Sciences, USA and R&D, USA).

HOMA-IR was calculated according to the following formula: fasting blood glucose (mmol/L)×fasting insulin (mU/L)/22.5.

Serum lipopolysaccharide binding protein (LBP), a marker of endotoxin load in blood, is considered as a central mediator in TLR4-mediated inflammatory responses. Adiponectin is an anti-inflammation and anti-diabetic hormone.

Tissue Inflammation Levels

Proinflammatory cytokine TNF-alpha plays a central role in inflammation, and is also involved in obesity and type 2 diabetes by inducing phosphorylation of Ser307 in insulin receptor substrate (IRS)-1. The adipose inflammatory response increases, prior to the inflammatory in other tissues (muscle and liver) and increase of fasting insulin level. Macrophages in adipose tissue play an active role in morbid obesity and insulin resistance. Monocyte chemoattractant protein (MCP)-1 is secreted by macrophage, which recruits additional macrophages to secrete large amounts of TNF-alpha and express CD11c in adipose tissue, then cause obesity and insulin resistance. CD11c+ cell depletion results in rapid normalization of insulin sensitivity. It is reported that adiponectin could inhibit chemokine production and the subsequent inflammatory responses, including infiltration of macrophages and release of proinflammatory cytokines in the mice.

Total RNA was extracted using RNeasy lipid tissue mini kit (QIAGEN), according to the manufacturer's instructions. RNA concentrations were measured using the Nanodrop Spectrophotometer and the integrity was checked by agarose gel electrophoresis. Contaminating DNA was removed using the DNase I (Invitrigen) digestion according to the manufacturer's instructions, and DNA contamination was tested by PCR with primer targeting housekeeping gene-GAPDH. Complementary DNA (cDNA) was randomly primed from 500 ng of high-quality total RNA using SuperScript III First-Strand synthesis system (Invitrogen).

Primer sequences for the Real-time PCR were as followed:

GAPDH:
(SEQ ID NO: 1)
F: GTGTTCCTACCCCCAATGTGT
(SEQ ID NO: 2)
R: ATTGTCATACCAGGAAATGAGCTT
TNF-α:
(SEQ ID NO: 3)
F: ACGGCATGGATCTCAAAGAC
(SEQ ID NO: 4)
R: AGATAGCAAATCGGCTGACG
CD11c:
(SEQ ID NO: 5)
F: CTGGATAGCCTTTCTTCTGCTG
(SEQ ID NO: 6)
R: GCACACTGTGTCCGAACTC
MCP-1:
(SEQ ID NO: 7)
F: TTAAAAACCTGGATCGGAACCAA
(SEQ ID NO: 8)
R: GCATTAGCTTCAGATTTACGGGT
adiponectin:
(SEQ ID NO: 9)
F: AGGTTGGATGGCAGGC
(SEQ ID NO: 10)
R: GTCTCACCCTTAGGACCAAGAA
leptin:
(SEQ ID NO: 11)
F: CCTGTGGCTTTGGTCCTATCTG
(SEQ ID NO: 12)
R: AGGCAAGCTGGTGAGGATCTG

The continuous amplification program consisted of one cycle at 95° C. for 4 min and then 40 cycles at 95° C. for 20 s, 55° C. for 30 s and 72° C. for 30 s, and finally one cycle at 94° C. for 15 s. The fluorescent products are detected in the last step of each cycle. Melting curve analysis was performed after amplification to distinguish the target from the non-targeted PCR products. The melting curve was obtained by slow heating at temperatures from 55 to 95° C. at a rate of 0.5° C./s with continuous fluorescence collection. Real-time PCR was subsequently performed using the iQ SYBR Green Surpermix (BIO-RAD) on a DNA Engine OPTICON2 continuous Fluorescence Detector (MJ research). Data were collected and analysed using MJ Opticon Monitor Analysis Software accompanying the PCR machine. All mRNA quantification data were normalized to GAPDH.

Gut Microbiota Composition

Genomic DNA was extracted from fecal sample by bead-beating extraction and InviMag Stool DNA Kit. The amount of DNA was determined by Fluorescent and Radioisotope Science Imaging Systems FLA-5100 (Fujifilm, Tokyo, Japan). Integrity of DNA was checked by 0.8% (w/v) agarose gel electrophoresis.

The V3 region of the 16S ribosomal RNA (rRNA) gene from each DNA sample was amplified using the bacterial universal primers:

(SEQ ID NO: 13)
F: 5′-NNNNNNNNCCTACGGGAGGCAGCAG-3′
and
(SEQ ID NO: 14)
R: 5′-NNNNNNNNATTACCGCGGCTGCT-3′

with a sample-unique 8-base barcode. PCR amplification, 454 pyrosequencing of the PCR amplicons, and quality control of data were performed as described previously (Zhang et al., 2010).

All reads were sorted into different samples according to barcodes. After removal of barcodes, the sequences were aligned by NAST multi-aligner with template length≧90 bases and percent identity≧75% (Greengenes) and then clustered using the program CD-HIT with 99.9% similarity. The most abundant sequence of each cluster was selected as a representative, and then imported into the ARB to construct a neighbour-joining tree. Operational taxonomic unit (OTU) was classified with Distance-Based OTU and Richness at 98% similarity level (DOTUR), and richness and diversity estimations were performed using Rarefaction analysis (aRarefact-Win software) and Shannon diversity index (H′) (R package 2.12.0). The most abundant sequence of each OTU (98% similarity) was inserted into pre-established phylogenetic trees of full-length 16S rRNA gene sequences in ARB for online Fast UniFrac analysis (unsupervised, considering the distance of the evolution) based on weighted (considering the abundance) and unweighted (not considering the abundance) metric. Relative abundances of OTUs were used for principal component analysis (unsupervised), multivariate analysis of variance (Matlab R2010a), and redundancy analysis (supervised) (Canoco for Windows 4.5). The representative sequence of each OTU was BLAST searched against the RDP database (RDP Classifier) at 50% confidence level to determine the phylogeny of the OUT, and relative abundances of different phyla and genera in each sample were calculated and compared between probiotic groups and HFD group using the Student's t-test (data of normalized distribution) or Mann-Whitney test (data of non-normalized distribution) via software SPSS 16.0.

### Results

HFD feeding induced obesity and insulin resistance in mice: compared with NC-fed mice, the HFD group showed higher body weight gain (FIG. 1A), elevated levels of fasting blood glucose (FBG) (FIG. 1B), fasting insulin (FINS) (FIG. 1C) and homeostasis assessment of insulin resistance (HOMA-IR) index (FIG. 1D), decreased glucose tolerance (FIGS. 1E and 1F). The supplement of probiotic strains B. lactis CNCM I-2494 or B. lactis B420 to HFD fed mice significantly decreased the body weight gain (FIG. 1A).

Although there was no significant difference in fasting blood glucose (FBG) and fasting insulin (FINS) levels between HFD+probiotic groups and HFD group, both probiotic strains B. lactis CNCM I-2494 and B. lactis B420 reduced the HOMA-IR index (FIG. 1D). Both probiotic strains B. lactis CNCM I-2494 and B. lactis B420 significantly decreased glucose intolerance (FIGS. 1E and 1F), indicating both probiotic strains could improve the insulin resistance.

The average energy intake per mouse per day (FIG. 2) was calculated for each of the twelve weeks of the trial. During all the trial, the energy intake of NC group was the lowest, and the energy intake of HFD+probiotic groups was almost the same with that of the HFD group except for the 7^{th} week. Cumulative energy intake of the four groups of animals during 3 months (FIG. 3) and cumulative energy intake of the four groups of animals during 12 weeks (FIG. 4) were calculated. This indicates that the body weight reduction observed for the probiotic treated groups cannot be attributed to a reduction of the energy intake.

The HFD-fed mice had significantly enhanced serum LBP level and lowered serum adiponectin concentration corrected for body weight than NC group. The serum LBP levels of both probiotic groups were not significantly lower than that of the HFD group, but HFD+CNCM I-2494 group had the lowest LBP level compared with HFD+B. lactis 420. Further, there were no significant differences between both probiotic groups and the NC group, which indicates that both probiotic strains tended to mitigate systemic antigen load. This indicates that probiotic strains B. lactis CNCM I-2494 and B. lactis B420 may improve insulin resistance through decreasing the serum LBP levels. Serum adiponectin corrected for body weight of both probiotic groups were all elevated compared with that of HFD group, however, the difference did not reach the statistical significance.

The impact of probiotics to the tissue inflammation levels in epididymal fat pad (eAT), liver and jejunum was measured. Levels of TNF-α, CD11c, MCP-1, adiponectin and leptin (another important proinflammatory adipokine) mRNA expression in eAT, and TNF-α mRNA expression in liver and jejunum were analyzed. High fat diet promoted the elevation of TNF-α and CD11c mRNA levels in eAT, and TNF-α mRNA expression in liver and jejunum, which suggested high fat diet induced inflammation in eAT, liver and jejunum. Probiotic strains B. lactis CNCM I-2494 and B. lactis B420 significantly reduced the TNF-α mRNA level in eAT compared with the HFD group. Both probiotic strains tended to reduce CD11c mRNA levels in eAT, because there were no significant differences between both probiotic groups and either the HFD group or NC group. MCP-1 mRNA levels in eAT in all of the four groups were not statistically significant different, while the level of HFD+CNCM I-2494 group was nearest to this of NC group. Similar to MCP-1 mRNA levels, there were not significant differences among the four groups, but HFD+CNCM I-2494 group showed increased adiponectin mRNA levels in eAT almost to equal to the level with NC group. Both probiotic groups did not decrease leptin mRNA levels in eAT compared with HFD group, suggesting that both probiotics did not decrease proinflammatory adipokine gene expression. The TNF-α mRNA levels in liver of the strain B. lactis B420 were not significantly different from either HFD group and NC group, which indicated they all tended to reduce TNF-α mRNA in liver, while B. lactis CNCM I-2494 significantly decreased TNF-α mRNA levels in liver. There were not significant differences in TNF-α mRNA levels in jejunum between both probiotic groups and either HFD group or NC group, which indicated both probiotic strains tended to decrease inflammation in jejunum. Taken together, these results show that B. lactis CNCM I-2494 most effectively reduced local inflammation in liver, epididymal adipose tissue and jejunum, compared with the strain B. lactis B420.

The 454 pyrosequencing of fecal bacterial 16S rRNA genes was performed. Multivariate statistical analyses were performed to compare the integral structure of gut microbiota of all the samples at the beginning and at the end of the trial. The structure of gut microbiota of HFD+probiotic groups, HFD group and NC group at 3 month of probiotics intervention was compared. Analysis of variance (ANOVA) was performed to compare the abundance of OTUs among individual HFD+probiotic groups, HFD group and NC group, and 111, 101, 95 and 99 OTUs were identified respectively, that were significantly changed. Then principal component analysis (PCA) based on the relative abundance of these OTUs revealed a separation of animals fed on HFD (including HFD group and individual HFD+probiotic groups) and the animals fed on NC, and the separation of HFD group and the individual HFD+probiotic groups mainly. Multivariate analysis of variance (MANOVA) test of PCA showed that there were significant differences among NC group, HFD group and each of both HFD+probiotic groups. These results suggest that probiotics change the structure of gut microbiota.

Partial least square discriminate analysis (PLS-DA), one supervised multi-variate statistical method, was used to identify key phylotypes of the gut microbiota whose abundance were changed by probiotics treatment. PLS-DA models were constructed to compare the bacterial composition between HFD-feeding (including both HFD group and HFD+probiotic groups) and NC-feeding animals, and between individual HFD+probiotic groups and the HFD group, and leave one-out cross-validation yielded high prediction rates for all the models. A total of 50 OTUs were found to be different in abundance between normal chow-fed mice and high fat diet-fed mice. Most of high fat diet changing OTUs were belong to families as Porphyromonadaceae (15 OTUs), Lachnospiraceae (9 OTUs), Ruminococcaceae (7 OTUs) and Erysipelotrichaceae (8 OTUs). 13 and 16 OTUs were changed by the probiotic strains B. lactis B420 and B. lactis CNCM I-2494 respectively. Strain B. lactis B420 mainly elevated the abundance of OTUs belonging to Bifidobacterium (1 OTU) and Barnesiella (1 OTU), and reduced some OTUs belonging to Lachnospiraceae (2 OTUs. Strain B. lactis CNCM I-2494 mainly elevated the abundance of OTUs belonging to Porphyromonadaceae (2 OTUs), Allobaculum (1 OTU), Olsenella (1 OTU), Lactobacillus (1 OTU), Coprococcus (1 OTU), and some OTUs belonging to Lachnospiraceae (1 OTU), and reduced OTU belonging to Alistipes (1 OTU). These results suggest that the anti-obesity and anti-insulin resistance effects of both probiotic strains may partially be mediated by enhanced levels of lactate and acetate-producing bacteria, because most of the enhanced gut bacteria by the probiotics all produce acetate and lactate. Indeed, the end products of glucose metabolism of strains belonging to Allobaculum are predominantly lactic and butyric acid, those of Coprococcus are butyric, acetic acids and lactic acid, Bifidobacterium strains produce acetic acids and lactic acids, and Olsenella could produce lactic and acetic acids. The bacteria decreased by probiotics are mainly harmful/non-beneficial bacteria.

To assess the link between the structural changes of the gut microbiota induced by probiotics and host phenotype variations, the correlation between the abundance of OTUs that are changed by probiotics and host phenotypic parameters was performed with spearman correlation analysis. Bifidobacterium (1 OTU), Olsenella (1 OTU), Porphyromonadaceae (3 OTUs), Allobaculum (1 OTU), Lachnospiraceae (3 OTUs) and Coprococcus (1 OTU) had negative correlations with obesity, insulin resistance and inflammation, while Alistipes (1 OTU), Porphyromonadaceae (3 OTUs), Oscillibacter (1 OTU) and Lachnospiraceae (7 OTUs) showed positive correlations with them. So most of the OTUs changed by probiotics were the key bacteria closely associated with host health, further confirming that the prevention of obesity and insulin resistance by probiotics is partially mediated by modulation of these key bacteria, especially by enhancing lactate and acetate-producing bacterial. There were some OTUs strongly correlate (R>0.5 or R<−0.5) with host phenotypes. One OTU from Porphyromonadaceae, accounting for 0.48±0.09% of total bacteria in each sample, showed negative correlation with weight gain (r=−0.54, p<0.001) and glucose intolerance (r=−0.52, p<0.001). There were negative correlations between one OTU from Allobaculum and weight gain (r=−0.51, p=0.030) and inflammation in liver (r=−0.51, p<0.001), and this OTU was a dominant OTU accounting for 2.28±0.52% of total bacteria in each sample. One OTU from Oscillibacter was positively associated with body weight gain (r=0.51, p=0.002), which accounting for 2.05±0.19% of total bacteria in each sample. One OTU from Lachnospiraceae showed positive correlation with glucose intolerance (r=0.64, p<0.001), and it could reach 3.30±0.45% of total bacteria in each sample. Another OTU from Porphyromonadaceae, which is significantly and negatively associated with inflammatory tone, was specifically enhanced by B. lactis CNCM I-2494. Hence, alleviation of inflammation by B. lactis CNCM I-2494 is associated with Porphyromonadaceae.

### REFERENCES

Aguirre, V., et al. (2001) J. Biol. Chem. 277:1531-1537.
Alberti, K., et al. (2005) Lancet 366:1059-1062.
Amar, J., et al. (2011) EMBO Mol. Med. 3:559-572.
Andreasen, A. S., et al. (2010) Br. J. Nutr. 104:1831-1838.
Backhed, F., et al. (2004) Proc. Natl. Acad. Sci. U.S.A. 101:15718-15723.
Backhed, F., et al. (2007) Proc. Natl. Acad. Sci. U.S.A. 104:979-984.
Cani, P. D., et al. (2008) Diabetes 57:1470-1481.
Fernandez, E. M., et al. (2011) Gut 60:1050-1059.
Ferrannini E, et al. (1998) J. Hypertens. 16:895-906.
Jia, W., et al. (2008) Nat. Rev. Drug. Discov. 7:123-129.
Larsen, N., et al. (2010) PLoS ONE 5:e9085.
Lee, H. Y., et al. (2006) Biochim. Biophys. Acta. 1761:736-744.
Ley, R. E., et al. (2006) Nature 444:1022-1023.
McNulty, N. P., et al. (2011) Sci Transl Med. 3: 106ra106.
Menard, S. (2004). Gut 53:821-828.
Turnbaugh, P. J., et al. (2006) Nature 444:1027-1131.
Veiga, P., et al. (2010) Proc. Natl. Acad. Sci. U.S.A. 107:18132-18137.
Vijay-Kumar, M., et al. (2010) Science 328:228-231.
Xu, R Y., et al. (2012) J. Clin. Biochem. Nutr. 50:72-77.
Yin, Y. N., et al. (2010) World J Gastroenterol. 16:3394-3401.
Zhang, C. H., et al. (2010) Isme Journal 4: 232-241.
Zhao, L. and J. Shen (2010) J Biotechnol. 149:183-190.

1. A composition comprising Bifidobacterium animalis subsp. lactis strain deposited at the collection nationale de cultures de micro-organismes (CNCM) with accession number I-2494, wherein the Bifidobacterium animalis subsp. lactis strain is present in an effective amount to decrease diet-induced body weight gain and diet-induced insulin resistance in a subject.

2. The composition of claim 1, wherein said diet-induced body weight gain and diet-induced insulin resistance are induced by a high fat diet.

3. The composition of claim 1, wherein Bifidobacterium animalis subsp. lactis strain treats, prevents or alleviates a condition resulting from diet-induced body weight gain and diet-induced-induced insulin resistance in the subject.

4. The composition of claim 3, wherein said condition is selected from the group consisting of overweight, obesity, and obesity-related disorders.

5. The composition of claim 1, wherein the composition is an orally administrable composition.

6. The composition of claim 5, wherein said composition is a food product or a food supplement.

7. The composition of claim 5, wherein said composition is a fermented dairy product.

8. A method of decreasing diet-induced body weight gain and improving diet-induced insulin resistance in a subject, comprising administering to the subject a composition comprising Bifidobacterium animalis subsp. lactis strain deposited at the collection nationale de cultures de micro-organismes (CNCM) with accession number I-2494.

9. The method of claim 8, wherein said body weight gain and insulin resistance are induced by a high fat diet.

10. The method of claim 8, wherein said composition is an orally administrable composition.

11. The method of claim 8, wherein said composition is a food product or a food supplement.

12. The method of claim 10, wherein said composition is a fermented dairy product.

13. The method of claim 12, wherein said fermented dairy product is a yogurt.

14. The method of claim 8, wherein the method treats, prevents or alleviates a condition resulting from diet-induced body weight gain and diet-induced-induced insulin resistance in the subject.

15. The method of claim 14, wherein said condition is selected from the group consisting of overweight, obesity, and obesity-related disorders.

16. The composition of claim 7, wherein the fermented dairy product is a yogurt.


SEQUENCE LISTING (14 sequences)

SEQ ID NO: 1 - DNA, 21 bp, Artificial Sequence
  Primer
gtgttcctacccccaatgtgt

SEQ ID NO: 2 - DNA, 24 bp, Artificial Sequence
  Primer
attgtcataccaggaaatgagctt

SEQ ID NO: 3 - DNA, 20 bp, Artificial Sequence
  Primer
acggcatggatctcaaagac

SEQ ID NO: 4 - DNA, 20 bp, Artificial Sequence
  Primer
agatagcaaatcggctgacg

SEQ ID NO: 5 - DNA, 22 bp, Artificial Sequence
  Primer
ctggatagcctttcttctgctg

SEQ ID NO: 6 - DNA, 19 bp, Artificial Sequence
  Primer
gcacactgtgtccgaactc

SEQ ID NO: 7 - DNA, 23 bp, Artificial Sequence
  Primer
ttaaaaacctggatcggaaccaa

SEQ ID NO: 8 - DNA, 23 bp, Artificial Sequence
  Primer
gcattagcttcagatttacgggt

SEQ ID NO: 9 - DNA, 16 bp, Artificial Sequence
  Primer
aggttggatggcaggc

SEQ ID NO: 10 - DNA, 22 bp, Artificial Sequence
  Primer
gtctcacccttaggaccaagaa

SEQ ID NO: 11 - DNA, 22 bp, Artificial Sequence
  Primer
cctgtggctttggtcctatctg

SEQ ID NO: 12 - DNA, 21 bp, Artificial Sequence
  Primer
aggcaagctggtgaggatctg

SEQ ID NO: 13 - DNA, 25 bp, Artificial Sequence
  Primer
nnnnnnnncctacgggaggcagcag

SEQ ID NO: 14 - DNA, 23 bp, Artificial Sequence
  Primer
nnnnnnnnattaccgcggctgct